(19) United States
(12) Patent Application Publication
(10) Pub. No.: US 2008/0243200 A1
(43) Pub. Date: Oct. 2, 2008

(54) METHODS AND APPARATUS FOR ENHANCING SPECIFICITY OF ARRHYTHMIA DETECTION USING FAR-FIELD SENSING AND INTRACARDIAC SENSING OF CARDIAC ACTIVITY

(76) Inventors: Anthony P. Scinicariello, Maple Grove, MN (US); Thomas H. Adamski, Andover, MN (US)

Correspondence Address: MEDTRONIC, INC., 710 MEDTRONIC PARKWAY NE, MINNEAPOLIS, MN, 55432-9924, US

(21) Appl. No.: 11/691,229

(22) Filed: Mar. 26, 2007

(51) Int. Cl. A61N 1/36

(52) U.S. Cl. 607/4

(57) ABSTRACT

Improved implantable medical devices (IMDS) and more particularly, a subcutaneous multiple electrode sensing and recording system for acquiring far- and near-field electrocardiographic (ECG) data and waveform tracings. The far-field ECG data and/or waveform tracings is used to confirm or refute sensing and detection performed by the near-field (e.g., epicardial and/or intracardiac) electrodes which collect electrograms (or EGMs). Thus, subcutaneously implanted devices adapted to sense near- and far-field cardiac activity offer improved specificity and sensitivity in arrhythmia sensing and detection. The far-field ECG signals are collected via at least a pair of electrodes that are directly mechanically coupled to the housing for the IMD (and thus spaced from the myocardium) which are filtered and processed and used in addition to the near-field EGM signals collected by lead-based electrodes.

### CROSS REFERENCE TO RELATED APPLICATIONS

The present patent document is related to co-pending non-provisional patent applications; namely, Ser. No. 11/085,843, entitled, “APPARATUS AND METHODS OF MONITORING CARDIAC ACTIVITY UTILIZING IMPLANTABLE SHROUD-BASED ELECTRODES,” filed on 22 Mar. 2005 and Ser. No. 11/380,811 entitled, “SHROUD-BASED ELECTRODES HAVING VENTED GAPS,” filed 28 Apr. 2006, the contents of which are hereby fully incorporated by reference herein. In addition, the contents of U.S. Pat. No. 7,151,962 entitled, “METHOD AND APPARATUS TO CONTROL DELIVERY OF HIGH-VOLTAGE AND ANTI-TACHY PACING THERAPY IN AN IMPLANTABLE MEDICAL DEVICE,” by Paul A. Belk is wholly incorporated as if set forth herein.

### FIELD OF THE INVENTION

The present invention relates generally to implantable medical devices (IMDs) and more particularly to a subcutaneous multiple electrode sensing and recording system for acquiring electrocardiographic data and waveform tracings from an implanted medical device (IMD). This data and/or waveform tracings are used to confirm or refute sensing and detection performed by epicardial and/or intracardiac electrodes (which generate electrograms, herein “EGMs”). More particularly, the present invention relates to subcutaneously implanted devices that are adapted to sense far-field cardiac activity via at least a pair of electrodes that are directly mechanically coupled to the housing for the IMD and thus spaced from the myocardium which are used in addition to lead-based electrodes that capture EGMs.

### BACKGROUND OF THE INVENTION

The electrocardiogram (ECG) is commonly used in medicine to determine the status of the electrical conduction system of the human heart. As practiced the ECG recording device is commonly attached to the patient via ECG leads connected to pads arrayed on the patient's body so as to achieve a recording that displays the cardiac waveforms in any one of 12 possible vectors.

Since the implantation of the first cardiac pacemaker, implantable medical device technology has advanced with the development of sophisticated, programmable cardiac pacemakers, pacemaker-cardioverter-defibrillator arrhythmia control devices and drug administration devices designed to detect arrhythmias and apply appropriate therapies. The detection and discrimination between various arrhythmic episodes in order to trigger the delivery of an appropriate therapy is of considerable interest. Prescription for implantation and programming of the implanted device are based on the analysis of the PQRST electrocardiogram (ECG) that currently requires externally attached electrodes and the electrogram (EGM) that requires implanted pacing leads. The waveforms are usually separated for such analysis into the P-wave and R-wave in systems that are designed to detect the depolarization of the atrium and ventricle respectively. Such systems employ detection of the occurrence of the P-wave and R-wave, analysis of the rate, regularity, and onset of variations in the rate of recurrence of the P-wave and R-wave, the morphology of the P-wave and R-wave and the direction of propagation of the depolarization represented by the P-wave and R-wave in the heart. The detection, analysis and storage of such EGM data within implanted medical devices are well known in the art. For example, S-T segment changes can be used to detect an ischemic episode. Acquisition and use of ECG tracing(s), on the other hand, has generally been limited to the use of an external ECG recording machine attached to the patient via surface electrodes of one sort or another.

The aforementioned ECG systems that utilize detection and analysis of the PQRST complex are all dependent upon the spatial orientation and number of electrodes available in or around the heart to pick up the depolarization wave front

As the functional sophistication and complexity of implantable medical device systems increased over the years, it has become increasingly more important for such systems to include a system for facilitating communication between one implanted device and another implanted device and/or an external device, for example, a programming console, monitoring system, or the like. For diagnostic purposes, it is desirable that the implanted device be able to communicate information regarding the device's operational status and the patient's condition to the physician or clinician. State of the art implantable devices are available which can even transmit a digitized electrical signal to display electrical cardiac activity (e.g., an ECG, EGM, or the like) for storage and/or analysis by an external device. The surface ECG, in fact, has remained the standard diagnostic tool since the very beginning of pacing and remains so today.

To diagnose and measure cardiac events, the cardiologist has several tools from which to choose. Such tools include twelve-lead electrocardiograms, exercise stress electrocardiograms, Holter monitoring, radioisotope imaging, coronary angiography, myocardial biopsy, and blood serum enzyme tests. Of these, the twelve-lead electrocardiogram (ECG) is generally the first procedure used to determine cardiac status prior to implanting a pacing system; thereafter, the physician will normally use an ECG available through the programmer to check the pacemaker's efficacy after implantation. Such ECG tracings are placed into the patient's records and used for comparison to more recent tracings. It must be noted, however, that whenever an ECG recording is required (whether through a direct connection to an ECG recording device or to a pacemaker programmer), external electrodes and leads must be used.

Unfortunately, surface electrodes have some serious drawbacks. For example, electrocardiogram analysis performed using existing external or body surface ECG systems can be limited by mechanical problems and poor signal quality. Electrodes attached externally to the body are a major source of signal quality problems and analysis errors because of susceptibility to interference such as muscle noise, power line interference, high frequency communication equipment interference, and baseline shift from respiration or motion. Signal degradation also occurs due to contact problems, ECG waveform artifacts, and patient discomfort. Externally attached electrodes are subject to motion artifacts from positional changes and the relative displacement between the skin and the electrodes. Furthermore, external electrodes require special skin preparation to ensure adequate electrical contact. Such preparation, along with positioning the electrode and attachment of the ECG lead to the electrode needlessly prolongs the pacemaker follow-up session. One possible approach is to equip the implanted pacemaker with the ability to detect cardiac signals and transform them into a tracing that is the same as or comparable to tracings obtainable via ECG leads attached to surface electrodes.

Previous art describes how to monitor electrical activity of the human heart for diagnostic and related medical purposes. U.S. Pat. No. 4,023,565 issued to Ohlsson describes circuitry for recording ECG signals from multiple lead inputs. Similarly, U.S. Pat. No. 4,263,919 issued to Levin, U.S. Pat. No. 4,170,227 issued to Feldman, et al, and U.S. Pat. No. 4,593,702 issued to Kepski, et al, describe multiple electrode systems, which combine surface EKG signals for artifact rejection.

The primary use for multiple electrode systems in the prior art is vector cardiography from ECG signals taken from multiple chest and limb electrodes. This is a technique whereby the direction of depolarization of the heart is monitored, as well as the amplitude. U.S. Pat. No. 4,121,576 issued to Greensite discusses such a system.

Numerous body surface ECG monitoring electrode systems have been employed in the past in detecting the ECG and conducting vector cardiographic studies. For example, U.S. Pat. No. 4,082,086 to Page, et al., discloses a four electrode orthogonal array that may be applied to the patient's skin both for convenience and to ensure the precise orientation of one electrode to the other. U.S. Pat. No. 3,983,867 to Case describes a vector cardiography system employing ECG electrodes disposed on the patient in normal locations and a hex axial reference system orthogonal display for displaying ECG signals of voltage versus time generated across sampled bipolar electrode pairs.

With regard to various aspects of time-release of surface coatings and the like for chronically implanted medical devices, the following issued patents are incorporated herein by reference. U.S. Pat. No. 6,997,949 issued 14 Feb. 2006 and entitled, “Medical device for delivering a therapeutic agent and method of preparation,” and U.S. Pat. No. 4,506,680 entitled, “Drug dispensing body implantable lead.” In the former patent, the following is described (from the Abstract section of the '949 patent) as follows: A device useful for localized delivery of a therapeutic agent is provided. The device includes a structure including a porous polymeric material and an elutable therapeutic agent in the form of a solid, gel, or neat liquid, which is dispersed in at least a portion of the porous polymeric material. Methods for making a medical device having blood-contacting surface electrodes is also provided.

Moreover, in regard to subcutaneously implanted EGM electrodes, the aforementioned Lindemans U.S. Pat. No. 4,310,000 discloses one or more reference sensing electrode positioned on the surface of the pacemaker case as described above. U.S. Pat. No. 4,313,443 issued to Lund describes a subcutaneously implanted electrode or electrodes for use in monitoring the ECG. Finally, U.S. Pat. No. 5,331,966 to Bennett, incorporated herein by reference, discloses a method and apparatus for providing an enhanced capability of detecting and gathering electrical cardiac signals via an array of relatively closely spaced subcutaneous electrodes (located on the body of an implanted device).

### SUMMARY

The present invention provides a leadless subcutaneous (or submuscular) multiple-electrode array that provides various embodiments of a compliant surround shroud directly coupled to a portion of an implantable medical device (IMD). The shroud incorporates a plurality of substantially planar electrodes mechanically coupled within recessed portions of the shroud. These electrodes electrically couple to circuitry of an IMD and are adapted to detect cardiac activity of a subject. Temporal recordings of the detected cardiac activity are referred to herein as an extra-cardiac electrogram (EC-EGM). The recordings can be stored upon computer readable media within an IMD at various resolution (e.g., continuous beat-by-beat, periodic, triggered, mean value, average value, etc.). Real time or stored EC-EGM signals can be provided to remote equipment via telemetry. For example, when telemetry, or programming, head of an IMD programming apparatus is positioned within range of an IMD the programmer receives some or all of the EC-EGM signals.

Electrode arrays according to the invention provide added specificity during sensing and detection of diverse cardiac events that are recorded by traditional transvenously-deployed endocardial- and epicardial-based electrodes. The present invention provides improved apparatus and methods for reliably collecting far-field EC-EGM signals for use in conjunction with near-field EGM signals to improve the specificity and sensitivity of arrhythmia detection in an IMD. A variety of different types of IMDs can benefit from the present invention, including without limitation, implantable pacemakers, implantable cardioverter-defibrillators or ICDs, subcutaneous ICDs, submuscular ICDs, and the like).

The invention employs suitable sensing amplifiers, switching circuits, signal processors, and memory to process the far-field EC-EGM signals and the near-field EGM signals between selected pair or pairs of the electrodes. The far-field electrodes are deployed in an array around the periphery or surface of a housing of an IMD to provide a leadless, orientation-insensitive means for receiving the EC-EGM signals from the heart. The near-field electrodes can be implemented in any convenient manner as is well-known in the art.

The shroud for the far-field electrodes can comprise a non-conductive, bio-compatible material such as any appropriate resin-based material, urethane polymer, silicone, or relatively soft urethane that retains its mechanical integrity during manufacturing and prolonged exposure to body fluids. Also, in lieu of a shroud discrete electrodes can be disposed on a localized insulative member or otherwise electrically insulated from the housing of an IMD. For instance, one or more of the electrodes can be coupled to the resin-based connector (or header) member of an IMD.

The shroud placed around the peripheral portions of an IMD can utilize a number of configurations (e.g., two, three, four recesses) for individual electrodes. However, a three-electrode embodiment appears to provide an improved signal-to-noise ratio. In one form of this embodiment the electrodes are located with approximately equal spacing therebetween (i.e., in an equilateral triangular configuration). And, embodiments having a single electrode pair appear much more sensitive (i.e., negatively) to appropriate orientation of the device relative to the heart than embodiments having more than a single pair of electrodes. Of course, embodiments of the invention using more than three electrodes increases complexity without providing a significant improvement in signal quality.

Embodiments having electrodes connected to three sense-amplifiers that are hardwired to three electrodes can record simultaneous EC-EGM signals. Alternative embodiments employ electrodes on the face of the lead connector, or header module, and/or major planar face(s) of the pacemaker that may be selectively or sequentially coupled in one or more pairs to the terminals of one or more sense amplifiers to pick up, amplify, filter and process the EC-EGM signals across each electrode pair. In one aspect, the EC-EGM signals from a first electrode pair are stored and compared to other electrode pair(s) in order to determine the optimal sensing vector. Following such an optimization procedure, the system can be programmed to chronically employ the selected subcutaneous EC-EGM signal vector.

For mass production of assemblies according to the invention a unique electrode piecepart can be fabricated for each unique conductor pathway and recess shape and configuration (including any of the variety of diverse mechanical interlocking features described hereinabove). Besides manufacturing processes such as metal stamping, the metallic electrode member(s) can be fabricating using electron discharge machining (EDM), laser cutting, or the like. It is desirable that the electrode assemblies are pre-configured (at least in a two-dimensional manner) so that little or no mechanical deformation or bending is required to fit each assembly into a shroud member. In addition, due to pre-configuring the parts the bends occur in a highly predictable manner and retain relatively little, if any, energy due to the spring-constant of the metal used to form the parts. In the event that electrical insulation or a dielectric layer becomes necessary or desirable, the major elongated portion of an electrode assembly can be coated with an insulative material such as paralyne or similar while the portions of the assembly likely to contact body fluid can be coated with diverse coatings pursuant to various embodiments of the invention.

Electrode assemblies according to the invention can be used for chronic or acute extra-cardiac electrogram (EC-EGM) signal sensing collection and attendant heart rate monitoring, capture detection, arrhythmia detection, and the like as well as detection of myriad other cardiac insults (e.g., ischemia monitoring using S-T segment changes, pulmonary edema monitoring based upon impedance changes).

In addition, the surface of the electrode can be treated with one or more electrode coatings to enhance signal-conducting, de- and re-polarization sensing properties, and to reduce polarization voltages (e.g., platinum black, titanium nitride, titanium oxide, iridium oxide, carbon, etc.). That is, the surface area of the electrode surfaces may be increased by techniques known in the art, and/or can be coated with such materials as just described and equivalents thereof. All of these materials are known to increase the true electrical surface area to improve the efficiency of electrical performance by reducing wasteful electrode polarization, among other advantages.

Many of the embodiments of the inventive electrodes herein can provide a continuous electrical path free of welds or bonds on a portion of the planar electrode, the transition portion, the elongated conductor or the distal tip portion. Moreover, the electrode assembly according to the invention anchors to a shroud member free of any chemical or adhesive bonding materials that can cause excursions due to electro-active specie release to the electrode surface or portions thereof.

These and other advantageous aspects of the invention will be appreciated by those of skill in the art after studying the invention herein described, depicted and claimed. In addition, persons of skill in the art will appreciate insubstantial modifications of the invention that are intended to be expressly covered by the appended claims.

### BRIEF DESCRIPTION OF THE DRAWINGS

FIG. 1 is an elevational side view depicting an exemplary shroud assembly coupled to an IMD which illustrates electrical conductors disposed in the header, or connector, portion of the IMD which is configured to receive a proximal end portion of medical electrical leads (not shown).

FIG. 2 is a perspective view of the IMD depicted in FIG. 1 further illustrating the shroud assembly.

FIG. 3 is a perspective view of an opposing major side of the IMD depicted in FIGS. 1 and 2.

FIG. 4 is a plan view of the IMD previously depicted that illustrates the relationship between two of the electrodes coupled to the shroud assembly as well as depicting the header, or connector, of the IMD.

FIG. 5 is a photocopy copy of a first side of a transparent shroud assembly coupled to a header according to the invention that clearly illustrates that the conductors and components of the assembly are readily visible.

FIG. 6 is a photocopy copy of a second side of the transparent shroud assembly coupled to a header according to the invention that clearly illustrates that the conductors and components of the assembly are readily visible from both sides.

FIG. 7 is a block diagram of an illustrative embodiment of an IMD in which the present invention may be employed.

FIG. 8 is a perspective view of an exemplary dual chamber IMD which can be utilized in conjunction with the present invention.

FIG. 9 is a perspective view of an exemplary triple chamber IMD which can be utilized in conjunction with the present invention.

### DETAILED DESCRIPTION OF THE DRAWINGS

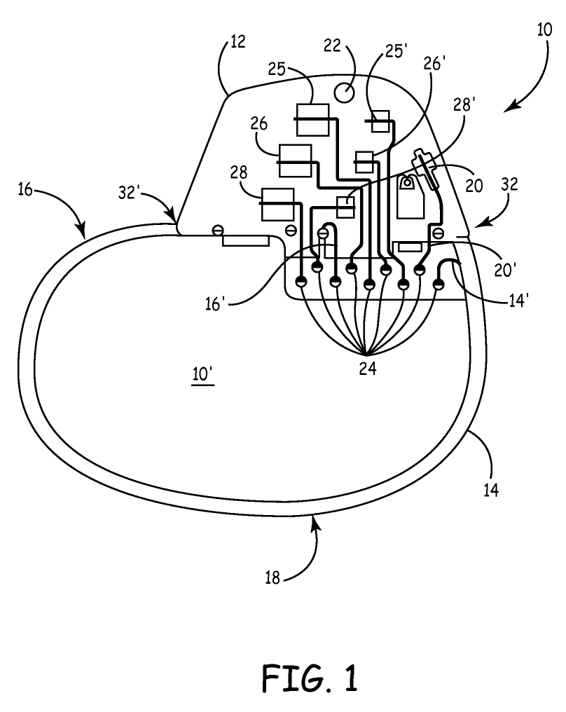

FIG. 1 is an elevational side view depicting an exemplary shroud assembly 14 coupled to an IMD 10 which illustrates electrical conductors 24,25,26,28 disposed in the header, or connector, portion 12 of the IMD 10 which are configured to couple to end portions of medical electrical leads as well as couple to operative circuitry within the IMD housing (not shown). The shroud assembly 14 surrounds IMD 10 and mechanically couples to the header portion 12 and includes at least three discrete electrodes 16,18,20 adapted for sensing far-field, or extra-cardiac electrogram (EC-EGM) signals. FIG. 1 also depicts an aperture 22 formed within the header 12 which can be used to receive thread used to suture the header 12 (and thus the IMD 10) to a fixed surgical location (also known as a pocket) of a patient's body.

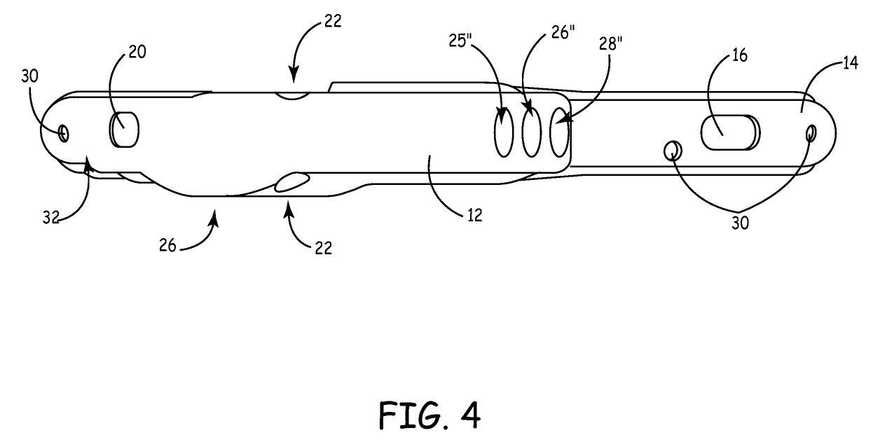

As partially depicted in FIG. 1, an elongated conductor 14′ couples to electrode 14, elongated conductor 16′ couples to electrode 16, and conductor segment 20′ couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12.

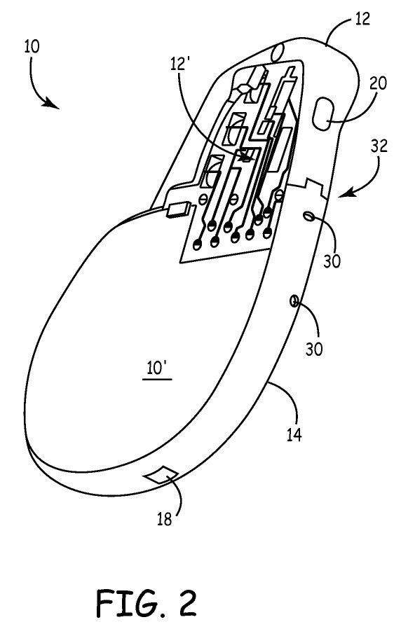

FIG. 2 is a perspective view of the IMD 10 depicted in FIG. 1 further illustrating the shroud assembly 14 and two of the three electrodes 18,20. In addition, two of a plurality of adhesive ports 30 and a mechanical joint 32 between the elongated portion of the shroud assembly 14 and the header 12 are also depicted in FIG. 2. The ports 30 can be used to evacuate excess medical adhesive disposed between the shroud assembly 14 and the IMD 10 and/or used to inject medical adhesive into one or more ports 30 to fill the void(s) therebetween. In one form of the invention, a major lateral portion 12′ of header 12 remains open to ambient conditions during assembly of the IMD 10. Subsequent to making electrical connections between the plurality of conductors of the shroud assembly 14 and the header 12, the open lateral portion 12′ is sealed (e.g., automatically or manually filled with a biocompatible substance such as a substantially clear medical adhesive, such as Tecothane® made by Noveon, Inc. a wholly owned subsidiary of The Lubrizol Corporation). Thus most if not all of the plurality of conductors of the shroud assembly 14 and the IMD 10 are visible and can be manually and/or automatically inspected to ensure long term operability and highest quality of the completed IMD 10.

Some properties of various Tecothane® appear below (as published in the Technical Data Sheet (TDS) for certain clear grades of the material:

Tecothane ® Typical Physical Test Date - CLEAR GRADES

| | ASTM Test | TT-1074A | TT-1085A | TT-1006A | TT-1056D | TT-1066D | TT-1060D | TT-1072D | TT-1075D-M |
|---|---|---|---|---|---|---|---|---|---|
| Durometer | D2240 | 75A | 85A | 94A | 54D | 64D | 68D | 74D | 75D |
| (Shore Hardness) | | | | | | | | | |
| Specific Gravity | D702 | 1.10 | 1.12 | 1.15 | 1.16 | 1.18 | 1.18 | 1.18 | 1.19 |
| Flexural Modulus | D790 | 1,300 | 3,000 | 8,000 | 18,000 | 26,000 | 44,000 | 73,000 | 180,000 |
| (psi) | | | | | | | | | |
| Ultimate Tensile | D412 | 6,000 | 7,000 | 9,000 | 9,600 | 10,000 | 9,800 | 9,000 | 8,300 |
| (psi) | | | | | | | | | |
| Ultimate Elongation | D412 | 550 | 450 | 400 | 350 | 300 | 310 | 275 | 150 |
| (%) | | | | | | | | | |
| Tensile (psi) | D412 | | | | | | | | |
| at 100% Elongation | | 500 | 900 | 1,300 | 2,500 | 2,800 | 3,200 | 3,700 | 3,600 |
| at 200% Elongation | | 700 | 1,000 | 2,100 | 3,800 | 4,600 | 4,200 | 3,900 | NA |
| at 300% Elongation | | 1,100 | 1,600 | 4,300 | 6,500 | 7,800 | NA | NA | NA |
| Melt Index | D1238 | 3.5 | 4.0 | 3.8 | 4.0 | 2.0 | 3.0 | 2.0 | 5.0 |
| (gm/10 min at | | (305° C.) | (305° C.) | (210° C.) | (210° C.) | (210° C.) | (210° C.) | (210° C.) | (210° C.) |
| 2160 gm load) | | | | | | | | | |
| Mold Shrinkage | D855 | .008-.012 | .008-.012 | .006-.010 | .004-.008 | .004-.008 | .004-.008 | .004-.005 | .004-.006 |
| (ln/$$) | | | | | | | | | |

Referring again to FIG. 1, the terminal ends of conductors 24 are depicted to include the optional shaped-end portion which provides a target for reliable automatic and/or manual coupling (e.g., laser welding, soldering, and the like) of the terminal end portions to respective conductive pins of a multi-polar feedthrough assembly (not shown). As is known in the art, such conductive pins hermetically couple to operative circuitry disposed within the IMD 10.

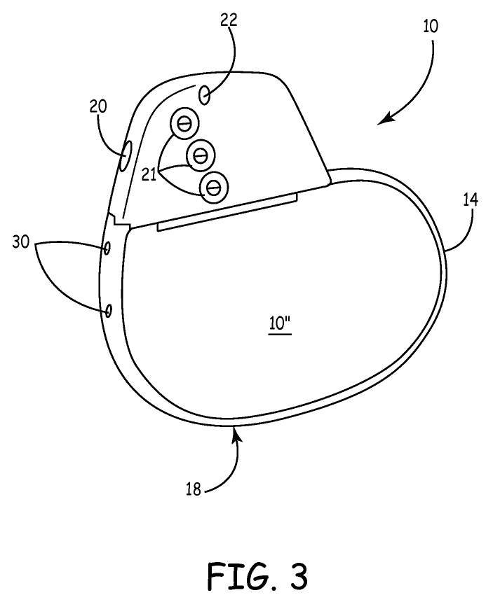

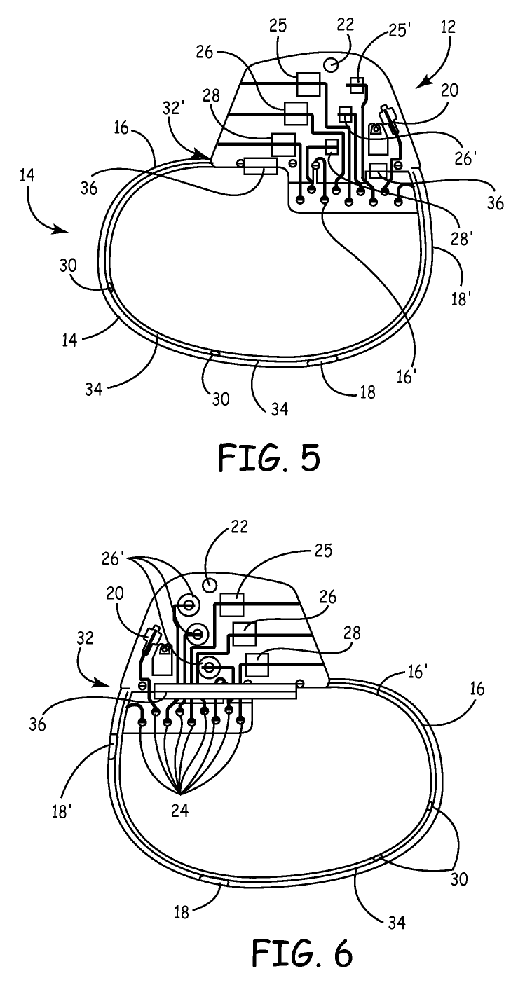

FIG. 3 is a perspective view of an opposing major side 10″ of the IMD 10 depicted in FIGS. 1 and 2 and three optionally self-healing grommets 21 substantially hermetically coupled to openings of a like number of threaded bores (shown in FIG. 6 and denoted by reference numeral 26′). As is known, the threaded bores are configured to receive a threaded shank and the grommets 21 are fabricated to temporarily admit a mechanical tool (not shown). The tool is used to connect and allow a physician or clinician to manually tighten the conductors 25,26,28 (depicted in FIGS. 5 and 6), for example, with compression and/or radially around conductive rings disposed on proximal portions of medical electrical leads (not shown). In addition, two of the plurality of ports 30 are also depicted in FIG. 3.

FIG. 4 is a plan view of the IMD 10 previously depicted that illustrates the relationship between two of the electrodes 16,20 coupled to the shroud assembly 14 as well as depicting the header 12, or connector, of the IMD 10. Opposing openings of the aperture 22 formed in the header 12 are also depicted in FIG. 4 as are the three openings 25″,26″,28″ of the bores or ports formed in the header 12 that are configured to admit the proximal end of medical electrical leads (not shown). Three of the adhesive-admitting ports 30 are shown distributed at various locations through the surfaces of the shroud 14.

Three elongated conductors individually couple to a respective electrode 16,18,20. These elongated conductors can be continuous or discrete segments of conductive material. In the event that they comprise discrete segments, they need to be coupled together such as with convention means like laser bonding, welding, soldering and the like. For example, the elongated conductor coupling to electrode 16 can traverse either direction around the periphery of the IMD 10 disposed within or mechanically coupled to an inner portion of the shroud 14. If it traverses past the seam 32 it might need to be isolated from the elongated conductor coupled to electrode 18 (assuming that conductor also traversed seam 32). If the conductor coupling electrode 16 is routed directly toward the header 12 (and the header/shroud is not a unitary structure) then a bond between segments of the elongated conductor could be necessary at the junction of the shroud 14 and the header 12.

FIG. 5 is a photocopy copy of a first side of a transparent shroud assembly 14 coupled to a header 12 according to the invention that clearly illustrates that the conductors and components of the assembly are readily visible. FIG. 6 is a photocopy copy of a second side of the transparent shroud assembly coupled to a header according to the invention that clearly illustrates that the conductors and components of the assembly are readily visible from both sides.

Since FIG. 5 and FIG. 6 essentially depict common components of the inventive assembly of the invention they shall be described together. The exemplary shroud assembly 14 of FIGS. 5 and 6 is depicted with an IMD 10 for clarity. The electrical conductors 25,26,28 disposed in the header, or connector, portion 12 of the IMD 10 are configured to couple to end portions of medical electrical leads as well as couple to operative circuitry within the IMD housing (not shown). The shroud assembly 14 mechanically couples to the header portion 12 at each end of the shroud assembly 14 both mechanically and electrically via medical adhesive (disposed at overlapping joint 32′) and an elongate conductor 16′ (passing through joint 32′). The three discrete electrodes 16,18,20 and their corresponding elongated conductors 16′,18′, 20′ are coupled together. While not depicted in FIGS. 5 and 6 the conductors 16′,18′,20′ have at least a partially serpentine configuration and conductors 16′,18′ are furthermore mechanically coupled to the shroud with a series of elongated stand-off bosses 34. In addition, and as previously mentioned, during attachment to an IMD adhesive is disposed intermediate the shroud 14 and the IMD with excess being evacuated from ports 30 (and/or if needed injected into one of more ports 30) to eliminate any air bubbles. Of course, one feature of the invention relates to the ability to fully inspect the finished article visually (including the quality of the electrical connections and the quality of the bond between the shroud 14 and an IMD. Also, the electrodes 16,18 can be at least one of mechanically embedded partially into the material of the shroud 14 and configured to receive medical adhesive to retain the electrodes in position (e.g., using perforated wing-like peripheral portions of the electrodes disposed at the ends, sides, and/or other parts of the periphery of an electrode). Aperture 22 also can be seen in FIGS. 5 and 6 formed in a peripheral portion of the header 12. Also depicted is how an elongated conductor couples to electrode 14, elongated conductor 16′ couples to electrode 16, and another conductor segment couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12. The joint 32 between header 12 and shroud 14 can comprise a variety of mechanisms, including an interlocking, partially spring-biased socket-type connection which, in combination with medical adhesive, provides a reliable mechanical coupling.

Another feature of the invention relates to including radio-opaque markers and/or identifiers within and/or on the shroud 14 so that a physician or clinician can readily determine that an IMD is outfitted with an assembly according to this invention. A marker according to this aspect of the invention can include a metallic insert and/or coating having a unique shape, location and/or configuration (e.g., an “M” or the corporate logo for an IMD manufactured by Medtronic, Inc.).

Depicted in FIGS. 5 and 6 is an elongated structural support member 36 which provides a reliable connection to a metallic housing of an IMD (not shown) via traditional processes (e.g., laser welding). The member 36 has a three substantially orthogonal sides (all denoted as 36 in FIGS. 5 and 6) thus providing three discrete bonding areas between the header 12 and an IMD. Of course, the member 36 could be perforated and/or coated with an insulative material, but in the embodiment depicted one side is cut out or not present so that the plurality of conductors 24 can pass from the header 12 and shroud 14 to the feedthrough array of the IMD.

Electrodes 16,18,20 and/or the (corresponding elongated conductors) can be fabricated out of any appropriate material, including without limitation tantalum, tantalum alloy, titanium, titanium alloy, platinum, platinum alloy, or any of the tantalum, titanium or platinum group of metals whose surface may be treated by sputtering, platinization, ion milling, sintering, etching, or a combination of these processes to create a large specific surface area. Also as noted herein, an electrode can be stamped, drawn, laser cut or machined using electronic discharge apparatus. Some of the foregoing might require de-burring of the periphery of the electrode or alternately any sharp edges due to a burr can be coupled facing toward the corresponding recess in the shroud member thereby minimizing likelihood of any patient discomfort post-implant while further reducing complexity in the fabrication of assemblies according to the invention. The electrodes can be coated or covered with platinum, a platinum-iridium alloy (e.g., 90:10), platinum black, titanium nitride or the like.

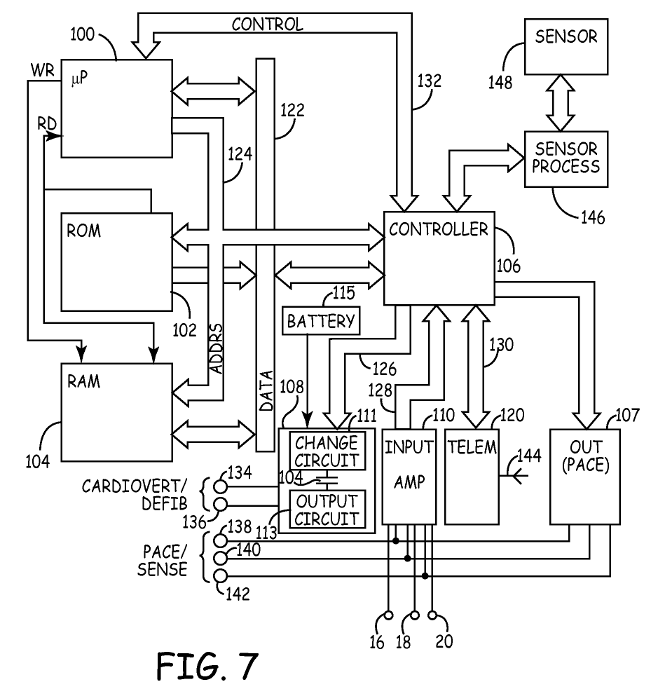

FIG. 7 is a block diagram of an illustrative embodiment of an IMD in conjunction with which the present invention may be employed. As illustrated in FIG. 7, the device is embodied as a microprocessor based stimulator. However, other digital circuitry embodiments and analog circuitry embodiments are also believed to be within the scope of the invention. For example, devices having general structures as illustrated in U.S. Pat. No. 5,251,624 issued to Bocek et al., U.S. Pat. No. 5,209,229 issued to Gilli, U.S. Pat. No. 4,407,288, issued to Langer et al, U.S. Pat. No. 5,662,688, issued to Haefner et al., U.S. Pat. No. 5,855,593, issued to Olson et al., U.S. Pat. No. 4,821,723, issued to Baker et al. or U.S. Pat. No. 4,967,747, issued to Carroll et al., all incorporated herein by reference in their entireties, may also be usefully employed in conjunction with the present invention. Similarly, while the device of FIG. 7 takes the form of a ventricular pacemaker/cardioverter, the present invention may also be usefully employed in a device having atrial pacing and cardioversion capabilities. FIG. 7 should thus be considered illustrative, rather than limiting with regard to the scope of the invention.

The primary elements of the IMD illustrated in FIG. 7 are a microprocessor 100, read-only memory (ROM) 102, random-access memory (RAM) 104, a digital controller 106, an input amplifier circuit 110, two output circuits 108 and 107, and a telemetry/programming unit 120. Read-only memory 102 stores the basic programming for the device, including the primary instruction set defining the computations performed to derive the various timing intervals employed by the cardioverter. RAM 104 generally serves to store variable control parameters, such as programmed pacing rate, programmed cardioversion intervals, pulse widths, pulse amplitudes, and so forth which are programmed into the device by the physician. Random-access memory 104 also stores derived values, such as the stored time intervals separating tachyarrhythmia pulses and the corresponding high-rate pacing interval.

Controller 106 performs all of the basic control and timing functions of the device. Controller 106 includes at least one programmable timing counter, which is initiated upon detection of a ventricular activation, and which times intervals thereafter. This counter is used to generate the basic timing intervals used to deliver anti-tachy pacing (ATP) pulses, and to measure other intervals used within for cardiac therapy delivery. On time-out of the pacing escape interval or in response to a determination that a cardioversion or defibrillation pulse is to be delivered, controller 106 triggers the appropriate output pulse from high-voltage output stage 108, as discussed below.

Following generation of stimulus pulses, controller 106 may be utilized to generate corresponding interrupts on control bus 132, waking microprocessor 100 from its “sleep” state, allowing microprocessor 100 to perform any required mathematical calculations, including all operations associated with evaluation of return cycle times and selection of anti-tachyarrhythmia therapies and the like. The timing/counter circuit in controller 106 also controls timing intervals such as ventricular refractory periods, as is known in the art. The time intervals may be determined by programmable values stored in RAM 104, or values stored in ROM.

Controller 106 also generates interrupts for microprocessor 100 on the occurrence of sensed ventricular depolarizations or beats. On occurrence of a sensed ventricular depolarization, in addition to an interrupt indicating its occurrence placed on control bus 132, the then-current value of the timing/counter within controller 106 is placed onto data bus 122. This value may be used by microprocessor 100 in determining whether a tachyarrhythmia is present, and further, in determining the intervals separating individual tachyarrhythmia beats.

Output stage 108 contains a high-output pulse generator capable of generating shock therapy to be applied to the patient's heart via electrodes 134 and 136, which are typically large surface area electrodes mounted on or in the heart, or located subcutaneously. Other electrode configurations may also be used, including two or more electrodes arranged within and around the heart. Typically the high output pulse generator includes one or more high-voltage capacitors 109, a charging circuit 111 for transferring energy stored in a battery 115 to the high-voltage capacitors 109, an output circuit 113 and a set of switches (not shown) to allow delivery of monophasic or biphasic cardioversion or defibrillation pulses to the electrodes employed.

In addition to output circuit 108, output circuit 107 is provided to generate pacing pulses. This circuit contains a pacing pulse generator circuit that is coupled to electrodes 138, 140 and 142, and which are employed to accomplish cardiac pacing, including ATP pacing pulses, by delivery of an electrical stimulation between electrode 138 and one of electrodes 140 and 142. Electrode 138 is typically located on the distal end of an endocardial lead, and is typically placed in the apex of the right ventricle. Electrode 140 is typically an indifferent electrode mounted on, or adjacent to, the housing of the cardioverter defibrillator. Electrode 142 may be a ring or coil electrode located on an endocardial lead slightly proximal to the tip electrode 138, or it may be another electrode positioned inside or outside the heart (i.e., epicardially). Although three electrodes 138 142 are shown in FIG. 7 for delivering pacing pulses, it is understood that the present invention may be practiced using any number of electrodes positioned in any pacing electrode configuration known in the art. Output circuit 108 may be controlled by control bus 126, which allows the controller 106 to determine the time, amplitude and pulse width of the pulse to be delivered. This circuit may also determine which electrode pair will be employed to deliver the pulse.

Sensing of ventricular depolarizations (beats) is accomplished by input amplifier 110, which couples to electrode 138 and one of electrodes 140 and 142 as well as the housing-based electrodes 16,18,20 according to the invention. Signals indicating both the occurrence of natural ventricular beats and paced ventricular beats are provided to the controller 106 via bus 128. Controller 106 passes data indicative of the occurrence of such ventricular beats to microprocessor 100 via control bus 132 in the form of interrupts, which serve to wake up microprocessor 100. This allows the microprocessor to perform any necessary calculations or to update values stored in RAM 104.

Optionally included in the device is one or more physiologic sensors 148, which may be any of the various known sensors for use in conjunction with implantable stimulators. For example, sensor 148 may be a hemodynamic sensor such as an impedance sensor as disclosed in U.S. Pat. No. 4,865,036, issued to Chirife or a pressure sensor as disclosed in U.S. Pat. No. 5,330,505, issued to Cohen, both of which are incorporated herein by reference in their entireties. Alternatively, sensor 148 may be a demand sensor for measuring cardiac output parameters, such as an oxygen saturation sensor disclosed in U.S. Pat. No. 5,176,137, issued to Erickson et al. or a physical activity sensor as disclosed in U.S. Pat. No. 4,428,378, issued to Anderson et al., both of which are incorporated herein by reference in their entireties. Sensor processing circuitry 146 transforms the sensor output into digitized values for use in conjunction with detection and treatment of arrhythmias.

External control of the implanted cardioverter/defibrillator is accomplished via telemetry/control block 120 that controls communication between the implanted cardioverter/pacemaker and an external device, such as a communication network or an external programmer, for example. Any conventional programming/telemetry circuitry is believed workable in the context of the present invention. Information entering the cardioverter/pacemaker from the programmer is passed to controller 106 via bus 130. Similarly, information from the cardioverter/pacemaker is provided to the telemetry block 120 via bus 130.

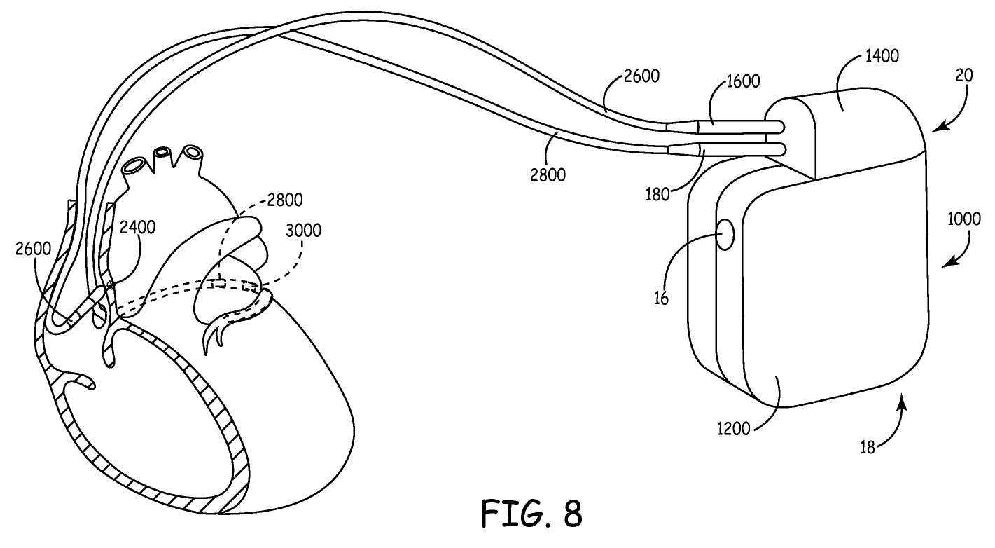

FIG. 8 illustrates an implantable pacemaker 1000 which can be used in accordance with the housing-based electrodes of the present invention and an associated lead set. The pacemaker comprises a hermetically sealed enclosure 1200 containing the pacemaker's circuitry and power source and carrying a connector block or header 1400 into which the connector assemblies 1800 and 1600 of two pacing leads 2000 and 2200 have been inserted. Pacing lead 2000 is a coronary sinus lead, and carries two electrodes 2800 and 3000 located thereon, adapted to be positioned adjacent the left atrium, within the coronary sinus/great vein of the patient's heart. Lead 2200 is a right atrial pacing lead carrying a distal, screw-in electrode 2400 and a proximal ring electrode 2600.

In conjunction with practicing the present invention, the pacemaker may employ the electrodes on the various leads in a variety of combinations. Multi-site pacing may be accomplished by simultaneously delivering pacing pulses to the right atrium using electrodes 2400 and 2600, with electrode 2400 serving as the pacing cathode and to the left atrium using electrodes 2800 and 3000, using either of electrodes 2800 and 3000 as the pacing cathode. Alternatively, multi-site pacing may be accomplished by delivering pacing pulses between electrodes 2400 and 3000 or between electrodes 2400 and 2800, with either of the two chosen electrodes serving as the cathode, in order to stimulate the right and left atria simultaneously by using electrode 2400 and either of to electrodes 2800 and 3000 as pacing cathodes and a conductive portion of the enclosure 1200 as a remote anode. Alternatively, the right atrium may be stimulated without stimulation of the left atrium by employing electrodes 2400 and 2600 or by employing electrode 2400 in conjunction with a conductive portion of the housing of the device enclosure 1200 to accomplish unipolar pacing. Similarly, pacing of the left atrium may be accomplished without corresponding pacing of the right atrium by pacing between electrodes 2800 and 3000 or by pacing between either of electrodes 2800 and 3000 and a conductive portion of the housing 1200.

The device 10 can be configured to allow the physician to program a prioritized list of tachyarrhythmia prevention pacing therapies and/or pacing sites and electrode configurations therein, for sequential application by the device 1000. For example, in the context of a device as illustrated in FIG. 8, the physician may request that the device 1000 initially delivers pacing pulses to the right and left atria between electrodes 2400 and 3000 as part of a first arrhythmia prevention therapy, with electrode 2400 being a cathodal electrode, delivers bipolar pacing pulses in the left atrium employing electrodes 2800 and 3000 as part of a second arrhythmia prevention therapy, with electrode 3000 being a cathodal electrode, and delivers bipolar pacing in the right atria employing electrodes 2400 and 2600 as part of a third arrhythmia prevention therapy, with electrode 2400 acting as a cathodal electrode. The first arrhythmia prevention therapy may, for example, simply be bi-atrial bradycardia pacing, while the second and third therapies may, for example, also include rate stabilization pacing as in the above-cited Mehra '471 patent.

Following programming, the device employs electrodes 2400 and 3000 to simultaneously pace both the right and left atria. Over the course of a defined extended time period of weeks or months, the device can detect a defined number and/or cumulative duration of tachyarrhythmias according to preset criteria. For example, a. tachyarrhythmia may be defined as a high atrial rate maintained for a minimum period of time. In response to each detected tachyarrhythmia episodes, the device can confirm said detection with reference to the shroud-based electrodes far-field sensing results. Thus, many available electrode configurations (i.e., vectors) can be used to reduce so-called false positive arrhythmia detections. Because diverse electrode configurations can be used and/or recorded, the real time performance and post-processing (review) of both EGM-sensed and far field-sensed events can be undertaken. If it is found that the device correctly detects a plurality of tachyarrhythmias during a given time period, the device has been appropriately programmed for a patient.

However, if the device records conflicting results from one or more possible arrhythmia episodes (as determined during review of the recordings of cardiac activity) other electrode sensing configurations can be employed. Once accurate detection of tachyarrhythmias takes place, the device can then remain in the appropriately programmed state. Otherwise, operation of the device in this fashion continues, with the choice of electrode configuration altered manually or automatically in response to an increase in the frequency of occurrence or cumulative duration of incorrectly-detected tachyarrhythmias, as compared to historical measurements of the accuracy compared to other electrode combinations.

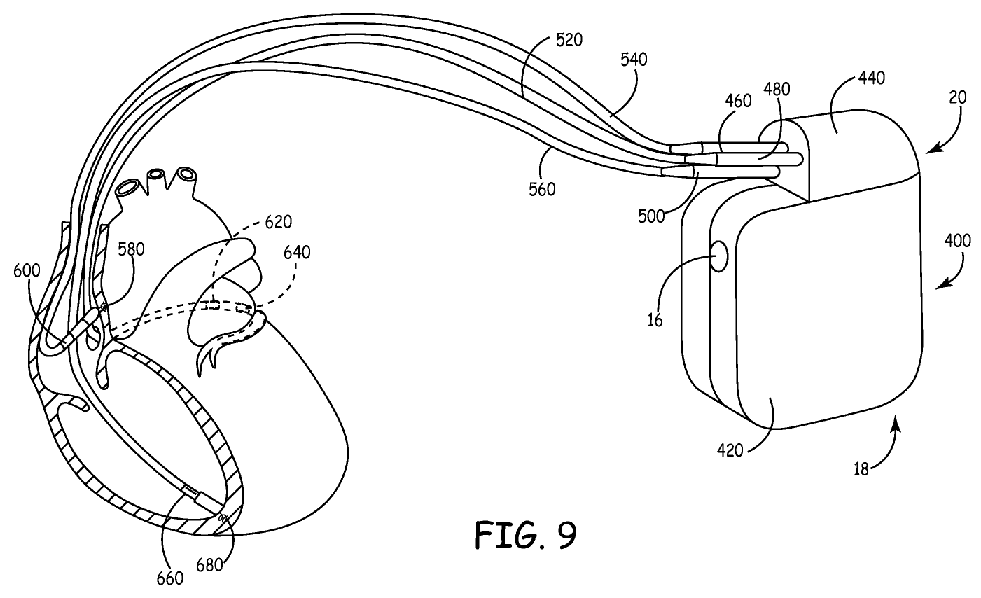

FIG. 9 illustrates an alternative embodiment of a pacemaker according to the present invention. Here the pacemaker 400 of FIG. 9 generally corresponds to the pacemaker 1000 of FIG. 8, with the addition of ventricular pacing capabilities. The pacemaker comprises a sealed hermetic enclosure 420 adapted to couple to the shroud and electrodes previously described containing the pacemaker's circuitry and power source and a connector block 440 which receives the connector assemblies 46, 48 and 50 of three pacing leads 520, 540 and 560. Leads 520 and 540 correspond to leads 2000 and 2200, respectively, of FIG. 8, and carry atrial pacing electrodes 580, 600, 620 and 640. Lead 560 is a ventricular pacing lead carrying a helical electrode 680 imbedded in the right ventricle of the heart and a ring electrode 660. A device according to FIG. 9 may employ multi-site atrial pacing in conjunction with ventricular pacing, using pacing modalities such as DDD, DVI and DDI pacing.

Accordingly, a number of embodiments and aspects of the invention have been described and depicted although the inventors consider the foregoing as illustrative and not limiting as to the full reach of the invention. That is, the inventors hereby claim all the expressly disclosed and described aspects of the invention as well as those slight variations and insubstantial changes as will occur to those of skill in the art to which the invention is directed. The following claims define the core of the invention and the inventors consider said claims and all equivalents of said claims and limitations thereof to reside squarely within their invention.

1. A subcutaneously implantable medical device (IMD), comprising:
A substantially hermetic housing for an implantable medical device (IMD);
a cardiac activity-sensing circuit disposed within the IMD housing;
a medical electrical lead adapted to couple to myocardial tissue of a heart;
a pair of electrodes adapted to couple to myocardial tissue and adapted to sense near-field cardiac activity via the cardiac-sensing circuit disposed within the IMD and provide a near-field signal therefrom;
a resilient shroud member adapted to cooperatively couple to at least part of the periphery of a subcutaneous IMD;
at least a pair of electrodes mechanically coupled to the shroud member and adapted to sense far-field cardiac activity via the cardiac-sensing circuit and provide a far-field signal therefrom; and
a processor coupled to said cardiac sensing circuit, wherein the processor is adapted to one of compare and store the near-field signal and the far-field signal and confirm or refute the detection of possible arrhythmia episodes based on said signals.

2. A device according to claim 1, further comprising a memory structure configured to store the respective output signals of the pair of electrodes and the at least a pair of electrodes.

3. A device according to claim 2, wherein the pair of electrodes are adapted to be disposed within the heart.

4. A device according to claim 3, wherein the at least a pair of electrodes include opposing major planar surfaces and the major planar surfaces mimic a curved portion of the resilient shroud member.

5. A device according to claim 4, wherein a first said opposing major planar surface has a greater surface area than a second said opposing major planar surface.

6. A device according to claim 5, wherein the first said opposing major planar surface couples to an interior surface portion of the shroud member and the second said opposing major plan surface is substantially coplanar with an exterior surface portion of the shroud member.

7. A device according to claim 6, further comprising a volume of substantially clear medical adhesive disposed between the interior surface portion of the shroud member and the periphery of the IMD.

8. A device according to claim 7, further comprising a plurality of ports formed between the interior surface portion and the exterior surface portion.

9. A shroud according to claim 1, further comprising a metallic bonding member coupled to the header portion and to a portion of the IMD.

10. A device according to claim 9, further comprising at least three spaced apart lead-coupling bores formed in the header portion.

11. A device according to claim 10, further comprising a pair of spaced apart conductors disposed within each of the at least three bores.

12. A device according to claim 1, further comprising a device connection module adapted to receive a proximal end portion of a medical electrical lead.

13. A device according to claim 12, wherein the module includes a suture-receiving aperture formed therethrough.

14. A device according to claim 1, wherein the at least a pair of electrodes are fabricated from one of a titanium material and a platinum material.

15. A device according to claim 14, wherein the at least a pair of electrodes further includes a coating on at least a major surface thereof.

16. A device according to claim 15, wherein the coating comprises one of a nitride coating, a carbon black coating, a time-release coating.

17. A device according to claim 1, further comprising medical grade adhesive disposed around between the at least a part of the periphery of the IMD.

18. A device according to claim 1, wherein the IMD comprises one of an implantable cardiac pacemaker and an implantable cardioverter-defibrillator.

19. A method, comprising:
receiving a signal of near-field cardiac activity in a subcutaneously implantable medical device (IMD);
receiving a signal of far-field cardiac activity in the subcutaneously IMD;
comparing the near-field signal and the far-field signal from a common temporal period; and
storing at least a portion of one of said near-field signal and said far-field signal in a memory structure.

20. A method according to claim 19, wherein the IMD comprises one of an implantable cardiac pacemaker and an implantable cardioverter-defibrillator.
